(19) United States
(12) Patent Application Publication
(10) Pub. No.: US 2026/0335282 A1
(43) Pub. Date: Oct. 29, 2026

(54) NOVEL 3-ARYLIDENE-2-INDOLINONE DERIVATIVES FOR THERAPEUTIC USE

(71) Applicant: ISTANBUL UNIVERSITESI REKTORLUGU, Fatih/Istanbul (TR)

(72) Inventors: Mustafa Oral ÖNCÜL, Fatih/Istanbul (TR); Nilgün Lütfiye KARALI, Fatih/Istanbul (TR); Gökçe CIHAN ÜSTÜNDAG, Fatih/Istanbul (TR); Özge SOYLU ETER, Fatih/Istanbul (TR); Tugçe CINEK, Fatih/Istanbul (TR); Sehnaz BOLKENT, Fatih/Istanbul (TR); Füsün ÖZTAY, Fatih/Istanbul (TR); Serap SANCAR, Fatih/Istanbul (TR); Dürdane Serap KURUCA, Fatih/Istanbul (TR); Günes ÖZEN EROGLU, Fatih/Istanbul (TR)

(21) Appl. No.: 19/112,054

(22) PCT Filed: Sep. 15, 2023
(86) PCT No.: PCT/TR2023/050966

§ 371 Date: Mar. 14, 2025

(30) Foreign Application Priority Data
Sep. 15, 2022 (TR) 2022/014289

(51) Int. Cl. A61K 31/404

(52) U.S. Cl. CPC A61K 31/404

(57) ABSTRACT

The present invention relates to the cytotoxic activity and tyrosine kinase inhibitory effects of 1-benzyl-3-(1H-indol-3-yl-methylidene)-1,3-dihydro-2H-indol-2-one derivatives. The invention provides novel 1-benzyl-3-(1H-indol-3-ylmethylidene)-1,3-dihydro-2H-indol-2-one derivatives for use in the treatment of human breast, lung, pancreas, colon and/or kidney cancer.

### TECHNICAL FIELD

The present invention relates to the cytotoxic activity and tyrosine kinase inhibitory effects of 1-benzyl-3-(1H-indol-3-ylmethylidene)-1,3-dihydro-2H-indol-2-one derivatives. The invention provides novel 1-benzyl-3-(1H-indol-3-ylmethylidene)-1,3-dihydro-2H-indol-2-one derivatives for use in the treatment of human breast, lung, pancreas, colon and/or kidney cancer.

### BACKGROUND OF THE INVENTION

Cancer is a complex disease caused by the uncontrolled division and proliferation of cells and develops under the influence of genetic and environmental conditions. With the advancement of technology, new treatment methods are being developed in addition to existing treatments. In addition to the standard chemotherapy, radiotherapy and surgical methods, the use of vaccines, also biological, hormonal, targeted and gene therapies is increasing.

Drugs used in cancer treatment are generally non-specific, causing DNA damage and cell stress leading to cell death. Therefore, target-specific drugs with nanomolar binding affinity are needed. Successful treatments and minimization of side effects are possible with the development of drugs selective to enzyme or receptor systems. This approach increases the sensitivity of cancer cells to chemotherapeutic drugs and therefore allows lower doses to be achieved. In this way, it will also be possible to reduce the side effects of chemotherapeutic drugs.

Suppression of disease-specific related enzyme or receptor mechanisms is one of the main targets of drug development research against many diseases, including cancer and immune diseases. Tyrosine kinases, which play a key role in cellular processes such as the cell proliferation, cel migration and survival, have become the target of researchers with the discovery that tyrosine kinase activity is increased in tumor cells. In line with this target, tyrosine kinases that catalyze their phosphorylation in the cell have been targeted and many 2-indolinone derivatives have been developed as tyrosine kinase inhibitors.

Tyrosine kinases are a large family of enzymes grouped into two classes: membrane-bound receptor tyrosine kinases (RTKs) and intracellular tyrosine kinases. RTKs are cell surface receptors to which many growth factors (epidermal growth factor, EGF; platelet-derived growth factor, PDGF; vascular endothelial growth factor, VEGF; fibroblast growth factor, FGF), cytokines and hormones bind with high affinity. The region on the cytoplasmic surface of RTKs shows tyrosine kinase activity. Upon ligand binding to the receptor, RTKs phosphorylate the tyrosine residues of target proteins inside the cell using ATP. Tyrosine kinases initiate the transduction of intracellular signals by moving phosphate from ATP to tyrosine in many specific substrate proteins. Growth factors associated with carcinogenesis play an important role in the regulation of signaling pathways. These target proteins are involved in important biological processes such as cell growth, proliferation, differentiation, motility, metabolism and programmed cell death (Du, Z., Lovly, C M., 2018, “Mechanisms of receptor tyrosine kinase activation in cancer”, Molecular Cancer, 17, 58).

The study performed by Wang et al. in 2015 shows that the oxindole nucleus can be selected as a pharmacophore group in the development of anticancer agents and that fluorine atom substitutions to these compounds produce favorable results in activity (Wang, S. Y., Wang, L. J., Jiang, B., Wu, N., Li, X. Q., Luo, J., Wang, B. C., Zhang, R. S., Xu, Q., Shi, D. Y., 2015, Synthesis and biological evaluation of novel fluorinated anticancer agents incorporating the indolin-2-one moiety, RSC Advances, 5 (111), 91795-91801). In another study by Kumar et al. in 2018, it has been stated that C-3 and C-5 substitutions to the oxindole ring increased antitumor activity (Kumar, N. P., Vanjarl, Y., Thatlkonda, S., Pooladanda, V., Sharma, P., Sridhar, B., Godugu, C., Kamal, A., Shankaraiah, N., 2018, Synthesis of enamino-2-oxindoles via conjugate addition between α-azido ketones and 3-alkenyl oxindoles: Cytotoxicity evaluation and apoptosis inducing studies, Bioorganic & Medicinal Chemistry Letters, 28 (22), 3564-3573).

The 2-indolinone structure can also exist as a natural alkaloid in various plants. For example, indurubin, which is responsible for the antileukemic effect, derived from Indigo naturalis, which is for use in the treatment of chronic myeloid leukemia and found in a traditional Chinese medicine prescription, contains the 2-indolinone core (Hoessel, R., Leclerc, S., Endicott, J. A., Nobel, M. E., Lawrie, A., Tunnah, P., Leost, M., Damiens, E., Marie, D., Marko, D., Niederberger, E., Tang, W., Eisenbrand, G., Meijer, L., 1999, Indirubin, the active constituent ofa Chinese antileukaemia medicine, inhibits cyclin-dependent kinases, Nature Cell Biology, 1 (1), 60-7).

During drug design, substituted indoles are recognized as a “privileged scaffold” for a large number of pharmacologically active lead compounds due to their significant affinity for many receptors. One of the simplest indole derivatives, isatin (1H-indole-2,3-dione, 1), has led to analogs with a wide range of biological properties, including anti-cancer activity. Indirubin (2), the active ingredient of Danggui Longhui Wan, a traditional Chinese medicine preparation used to treat myelocytic leukemia, has been stated to be anti-proliferative against cancer cells by inhibiting genes or proteins that regulate cell cycle progression. The inhibited growth induced by indirubin-3-oxime (3) is associated with induction of the cyclin-dependent kinase inhibitor p21, inhibition of cyclin D1 and activation of caspase-3. Indirubin derivatives are stated to inhibit signal transducer and activator of transcription 3 signaling and induce apoptosis in cancer cells. 1H-indole-2,3-dione can be used as a privileged scaffold to design various therapeutic molecules, and many 1H-indole-2,3-dione derivatives have been studied as antitumor agents. Among the stated 1H-indole-2,3-dione derivatives, 5,7-dibromo-1-(naphthalen-1-ylmethyl)-1H-indole-2,3-dione (4) has potent anti-tumor properties with IC₅₀ (Li, P., Tan, Y., Liu, G., Liu, Y., Liu, J., Yin, Y., & Zhao, G. (2014). Synthesis and biological evaluation of novel indoline-2,3-dione derivatives as antitumor agents. Drug Discoveries & Therapeutics, 8(3), 110-116).

The chemical structures of these compounds (1-4) are shown below.

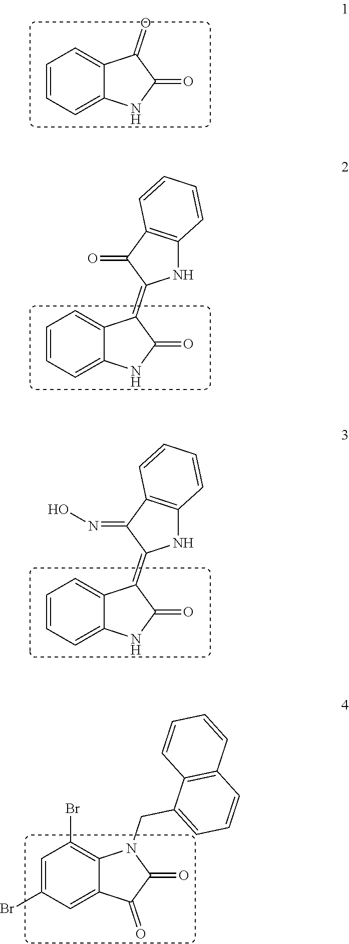 [SMILES: O=C1Nc2ccccc2C1=O ; O=C1Nc2ccccc2/C1=C1/Nc2ccccc2C1=O ; O=C1Nc2ccccc2/C1=C1/Nc2ccccc2/C1=N\O ; O=C1C(=O)N(Cc2cccc3ccccc23)c2c(Br)cc(Br)cc21]

Indolinone group kinase inhibitors have been first discovered in 1997 by Mohammadi et al. SU5402 and SU4984 are the first tyrosine kinase inhibitors with a 2-indolinone structure. These small molecules have low molecular weight and many of them bind to the ATP binding site in competition with ATP (Prakash, P., Theivendren, P., Raja, S., 2012, Indolin-2-one in clinical trials as potential kinase inhibitors: a review, Pharmacology & Pharmacy, 3, 62-71).

Among these compounds targeting the ATP binding site, SU4984, a 4-[4-[(Z)-(2-oxo-1H-indol-3-ylidene)methyl]phenyl]piperazine-1-carbaldehyde structure, targets the fibroblast growth factor receptor, SU5402, which is 3-[4-methyl-2-[(Z)-(2-oxo-1H-indol-3-ylidene)methyl]-1H-pyrrol-3-yl]propanoic acid, inhibits the vascular endothelial growth factor receptor (VEGFR). SU4984 targets the ATP binding site of FGFR1. It also inhibits fibroblast growth factor (FGF)-induced phosphorylation of extracellular signal-regulated kinase (ERK) 1 and ERK 2 and tyrosine phosphorylation of platelet-derived growth factor (PDGF) and insulin receptors. SU4984 is also active against stem cell growth factor receptor (KIT) with juxtamembrane activating mutations (Prakash, P., Theivendren, P., Raja, S., 2012, Indoline-2-one in clinical trials as potential kinase inhibitors: a review, Pharmacology & Pharmacy, 3, 62-71).

SU5402 is a tyrosine kinase inhibitor that inhibits VEGFR and FGFR1 in an ATP-competitive manner. It has been determined that the compound, which is one of the first generation FGFR tyrosine kinase inhibitors targeting the ATP binding site in the oxindol structure, shows better inhibitory activity against FGFR1 than SU4984 with its hydrogen bond interactions (Liang, G., Liu, Z., Wu, J, Cai, Y., Li, X., 2012, Anticancer molecules targeting fibroblast growth factor receptors, Trends in Pharmacological Sciences, 33, 531-541). Another oxindol derivative compound, SU6668 (orantinib), was developed by Pfizer as a multi-target RTK inhibitor and Phase 2 studies are performed on it due to its detected antitumor activity.

Mohammadi et al. suggested that more specific, ATP-competitive protein tyrosine kinase (PTK) inhibitors could be developed by substitutions at the 5 and 6 positions of the oxindol core, and it has been revealed that the 2-indolinone core is responsible for the activity (Mohammadi, M., Mcmahon, G., Sun, L., Tang, C., Hirth, P., Yeh, B. K., Hubbard, S. R., Schlessinger, J., 1997, Structures of the Tyrosine Kinase Domain of Fibroblast Growth Factor Receptor in Complex with Inhibitors, Science, 276(5314), 955-960).

The chemical structures of SU4984 and SU5402 compounds are shown below.

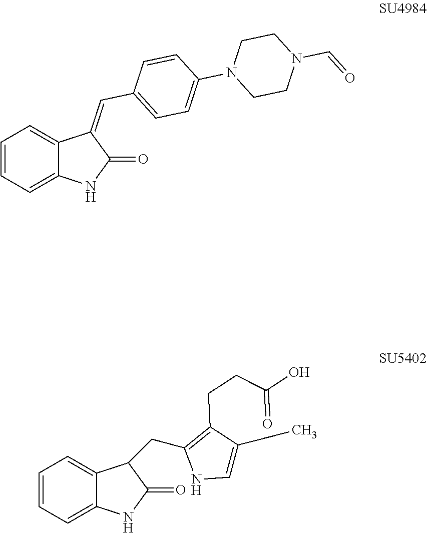 [SMILES: O=CN1CCN(c2ccc(/C=C3\C(=O)Nc4ccccc43)cc2)CC1 ; Cc1c[nH]c(CC2C(=O)Nc3ccccc32)c1CCC(=O)O]

As a result of various studies, it has been observed that the hydrophobic region of tyrosine kinase interacts with the OH groups of ATP sugar and N/NH groups of adenine via hydrogen bonds (Prakash, P., Theivendren, P., Raja, S., 2012, Indolin-2-one in clinical trials as potential kinase inhibitors: a review, Pharmacology & Pharmacy, 3, 62-71).

The addition of groups such as phenyl, imidazole, pyrrole to the 3-position of 2-indolinones increases the selectivity of RTKs and 3-substituted-2-indolinones have been reported to selectively and potently inhibit RTKs in the literature. In addition, they act as adenine-mimetics and bind to the ATP binding site (Prakash, P., Theivendren, P., Raja, S., 2012, Indolin-2-one in clinical trials as potential kinase inhibitors: a review, Pharmacology & Pharmacy, 3, 62-71).

Furthermore, the interactions of 3-substituted-2-indolinone tyrosine kinase inhibitors such as sunitinib and semaxanib in the ATP binding site of the enzyme have been examined and it has been revealed that the polar region of sunitinib is bonded by hydrogen bonds, and the hydrophobic region of both sunitinib and semaxanib is located in the hydrophobic pocket of the enzyme (Kaur, J, Kaur, B., Singh, P., 2018, Rational modification of semaxanib and sunitinib for developing a tumor growth inhibitor targeting ATP binding site of tyrosine kinase, Bioorganic & Medicinal Chemistry Letters, 28, 129-133).

The chemical structures of sunitinib and semaxanib are shown below.

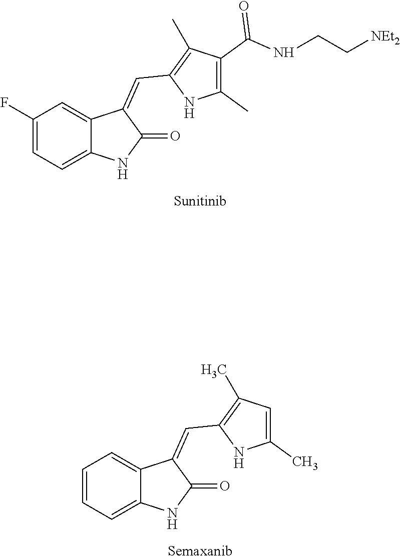

Semaxanib (SU5416) is the first VEGFR tyrosine kinase inhibitor tested in clinical trials (Prakash, P., Theivendren, P., Raja, S., 2012, Indolin-2-ones in clinical trials as potential kinase inhibitors: a review, Pharmacology & Pharmacy, 3, 62-71). Sunitinib has been developed to reduce the side effects of semaxanib after the phase studies have been ceased due to the side effects of semaxanib during the phase studies (Kaur, J., Kaur, B., Singh, P., 2018, Rational modification ofsemaxanib and sunitinib for developing a tumor growth inhibitor targeting ATP binding site of tyrosine kinase, Bioorganic & Medicinal Chemistry Letters, 28, 129-133).

Toceranib (SU11654), which has a similar structure to sunitinib, has potent inhibitory effects against kinase receptors such as VEGFR, PDGFR and c-Kit. The compound has received approval from the EMA for use in dogs for the treatment of mast cell tumors of the skin (Prakash, C. R., Raja, S., Theivendren, P. 2012. “Indolin-2-Ones in Clinical Trials as Potential Kinase Inhibitors: A Review” Pharmacology & Pharmacy, 3, 62-71). Structurally very similar to toceranib, SU14813 is another multikinase inhibitor targeting VEGFR, PDGFR, c-Kit and FLT3. Combination treatment of SU14813 with docetaxel was found to significantly improve both inhibition of primary tumor growth and survival of tumor-bearing mice (Patyna, S., Laird, A. D., Mendel, D. B., O'Farrell, A. M., Liang, C., Guan, H., Vojkovsky, T., Vasile, S., Wang, X., Chen, J., Grazzini, M., Yang, C. Y., Haznedar, J. O., Sukbuntherng, J, Zhong, W. Z., Cherrington, J. M., Hu-Lowe, D. 2006. “SU14813: a novel multiple receptor tyrosine kinase inhibitor with potent antiangiogenic and antitumor activity” Molecular Cancer Therapeutics, 5(7), 1774-1782).

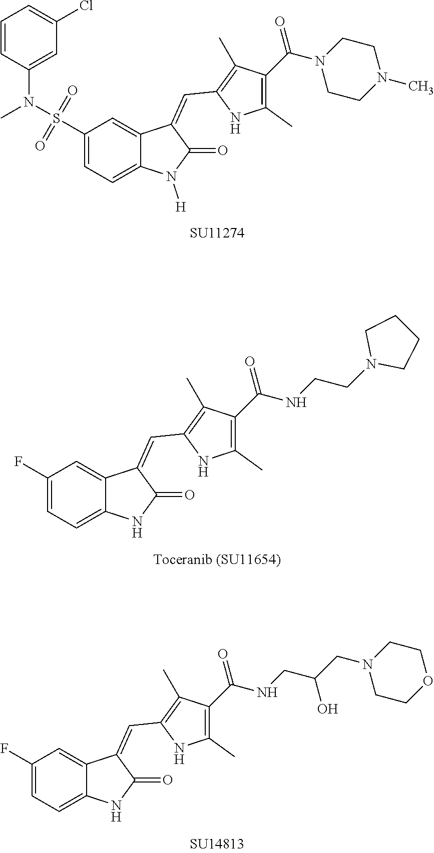

It is possible for cancer cells to become resistant to first-generation TKIs (Iderzorig, T., Kellen, J., Osude C., Singh, S., Woodman, J. A., Garcia, C., Puri, N. 2018. “Comparison of EMT mediated tyrosine kinase inhibitor resistance in NSCLC”, Biochemical and Biophysical Research Communications, 496(2), 770-7). Despite the availability of second and third generation TKIs that overcome acquired drug resistance, the use of current chemotherapeutics is often limited due to side effects in addition to drug resistance. There is an urgent need to discover more effective TKIs. Among the 3-arylidene-2-indolinone derivatives synthesized in recentyears, some of the compounds bearing an imidazothiazole ring have been identified as nontoxic, potent antiproliferative agents in the nM concentration ranges. In studies on HeLa, HT-29 and A549 cells, the compounds have been reported to induce apoptosis via the mitochondrial death pathway and cause both caspase-3 and caspase-9 activation, PARP cleavage and downregulation of anti-apoptotic proteins Bcl-2 and Mcl-1.

The 2-indolinone derivative compound (I), whose formula is given below, has been also studied in vivo in mouse BL6-B16 melanoma and E0771 breast cancer cells and has been found to cause a reduction in tumor volume (Morigi, R., Locatelli, A., Leoni, A., Rambaldi, M., Bortolozzi, R., Mattiuzzo, E., Ronca, R., Maccarinelli, F., Hamel, E., Bai, R., Brancale, A., Viola, G. 2019. “Synthesis, in vitro and in vivo biological evaluation of substituted 3-(5-imidazo[2,1-b]thiazolylmethylene)-2-indolinones as new potent anticancer agents”, European Journal of Medicinal Chemistry, 166, 514-530). In a recent study, some synthesized sunitinib derivatives substituted at the 1-, 5- or 6-positions of the indole ring have been tested for inhibition of AMP-activated protein kinase (AMPK) and the cellular interaction of AMPK with the compounds has been determined by inhibition of phosphorylation of Acetyl-CoA carboxylase (ACC), an AMPK substrate, in myeloid leukemia cells. Two derivatives (II) which is bearing a 2-hydroxyethyl or 3-amino-3-oxopropyl group at the 5-position of the indole ring have been found to be potent inhibitors of cellular AMPK at nanomolar concentration ranges and the compounds had not been show cytotoxicity in the K562 cell line (Matheson, C. J., Casalvieri, K. A., Backos, D. S., Minhajuddin, M., Jordan, C. T., Reigan, P. 2020. “Substituted oxindol-3-ylidenes as AMP-activated protein kinase (AMPK) inhibitors”. European Journal of Medicinal Chemistry, 197, 112316).

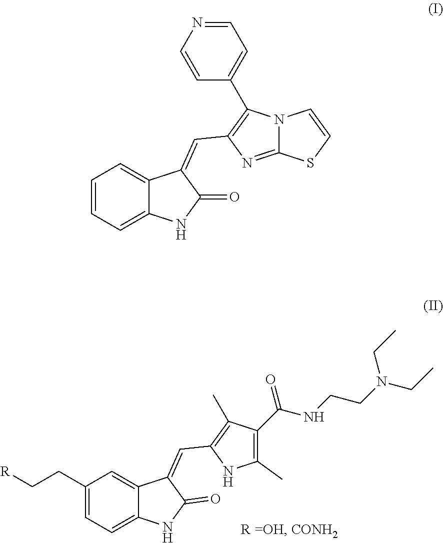

ZMF-005, a novel 2-indolinone derivative targeting oncoprotein p21-activated kinase 1 (PAK1) in breast cancer cells, has been described by Yao et al. in 2020. ZMF-005 has been reported to induce apoptosis and suppress migration in MDA-MB-231 cells (Yao, D., Ruhan, A., Jiang, J., Huang, J., Wang, J, Han, W. 2020. “Design, synthesis and biological evaluation of 2-indolinone derivatives as PAK1 inhibitors in MDA-MB-231 cells” Bioorganic & Medicinal Chemistry Letters, 30(17), 127355). In another study in 2021, the inhibitory effects of 2-indolinone derivatives bearing 1,2,3-triazole ring against VEGFR-2 have been investigated in vitro and in vivo models. The following compound (III) has been found to have greater inhibitory activity on VEGFR-2, better cytotoxicity on HT-29 and MKN-45 cells and less toxic to HUVEC cell line than sunitinib. Moreover, the inhibitory effect of this compound on angiogenesis has been also confirmed in vivo using a transgenic zebrafish model with fluorescently labeled VEGFR-2 gene (Wang, D., Liu, K, Li, X., Lu, G., Xue, W., Qian, X., Mohamed, K. O., Meng, F. 2021. “Design, synthesis, and in vitro and in vivo anti-angiogenesis study of a novel vascular endothelial growth factor receptor-2 (VEGFR-2) inhibitor based on 1,2,3-triazole scaffold”, European Journal of Medicinal Chemistry, 211, 113083).

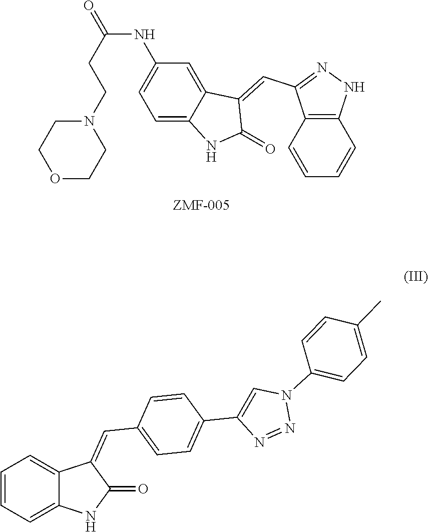 [SMILES: O=C(CCN1CCOCC1)Nc1ccc2c(c1)/C(=C/c1n[nH]c3ccccc13)C(=O)N2 ; Cc1ccc(-n2cc(-c3ccc(/C=C4\C(=O)Nc5ccccc54)cc3)nn2)cc1]

In another study published in 2020, the in vitro VEGFR-2 kinase inhibitory activity of compounds with the N-benzyl-2-indolinone skeleton (IV and V) has been examined and it has been observed that most of the compounds had IC₅₀ values (concentration that inhibits the proliferation of 50% of cells in vitro) below 1 μM, similar to sunitinib. The in vitro activity of the compounds against CAKI-1 and A498 renal tumor cells has been also reported to be comparable to sunitinib, while the selectivity index of some compounds against normal renal cell types was higher than sunitinib (Mahmoud, H. K, Farghaly, T. A., Abdulwahab, H. G., Al-Qurashi, N. T., Shaaban, M. R. 2020. “Novel 2-indolinone thiazole hybrids as sunitinib analogues: Design, synthesis, and potent VEGFR-2 inhibition with potential anti-renal cancer activity”, European Journal of Medicinal Chemistry, 208, 112752).

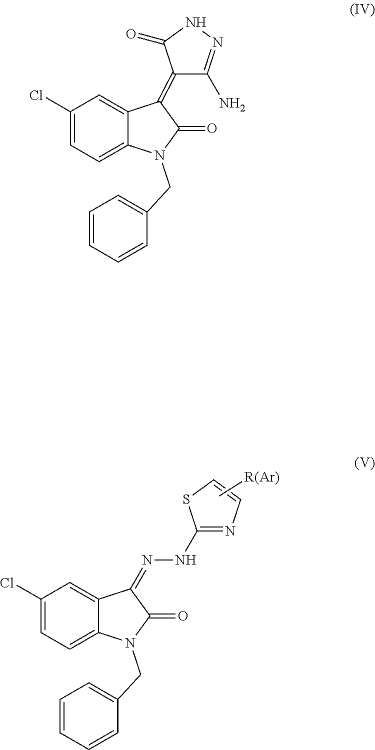

EP 1139754 A1 relates to 3-heteroaridenidenyl-2-indolinone compounds that modulate the activity of protein kinases (PKs) and their combination with other chemotherapeutic agents for use in the treatment of disorders related to abnormal protein kinase activity, including cancer. The document also states that these compounds are expected to enhance the efficacy of other chemotherapeutic agents, particularly fluorinated pyrimidines, in the treatment of cancer.

CA 2383623 A1 relates to novel 3-methylideneyl-2-indolinone compounds and their physiologically acceptable salts and prodrugs that modulate the activity of protein kinases and are thus expected to be useful in the prevention and treatment of protein kinase-related cellular disorders such as cancer.

CA 2726644 A1 relates to a pharmaceutical combination that can be useful for the treatment of diseases involving cell proliferation, displacement or apoptosis of myeloma cells, angiogenesis or fibrosis. The present invention also relates to a method for use in the treatment of said diseases, comprising the simultaneous, separate or sequential administration of effective amounts of specific active compounds and/or radiation therapy and/or concurrent treatment with an additive in a ratio providing a synergistic effect, and to the combined use of these specific compounds and/or radiation therapy for the production of corresponding pharmaceutical combination preparations. The invention relates to a pharmaceutical combination comprising 3-Z-[1-(4-(4-(N-((4-methyl-piperazin-1-yl)-methylcarbonyl)-N-methyl-amino)-anilino)-1-phenyl-methylene], -6-methoxycarbonyl-2-indolinone or a pharmaceutically acceptable salt thereof and N-[4-[4-[2-(2-amino-4,7-dihydro-4-oxo-1H-pyrrolo[2,3-d]pyrimidin-5-yl)ethyl]benzoyl]-L Glutamic acid or a pharmaceutically acceptable salt thereof.

EP 0161918 A2 relates to certain indoline and 2-indolinone derivatives, their pharmaceutical formulations and their use as positive inotropic agents. The document is about potent, long-lasting and orally effective positive inotropic agents that cause minimal effect on blood pressure and heart rate.

In the light of the above information, it can be seen that the 3-arylidene-2-indolinone derivatives SU-5416 (semaxanib), SU-11248 (sunitinib) and SU-9516 are tyrosine kinase inhibitors. Sunitinib, which selectively inhibits various receptor tyrosine kinases and has anticancer and antiangiogenic activity, was approved in 2006 for the treatment of advanced renal and gastrointestinal stromal cancers. 2-Structure-activity studies on indolinone derivatives have been shown that electron-withdrawing substituents at positions 5 and 7 in the indole ring and an aromatic ring with 1 carbon chain at position 1 increase activity against tumor cells.

2-indolinone compounds have been targeted tyrosine kinases that catalyze phosphorylation in cells for use in the cancer treatment and many 2-indolinone derivatives have been developed as tyrosine kinase inhibitors. Although the antitumor activity of 2-indolinone compounds for cancer treatment has been tested, there is no study investigating the anticancer activity of derivatized versions of these compounds. Therefore, there is a need for studies on new 3-arylidene-2-indolinone derivatives in the relevant technical field. Thus, the present invention provides cytotoxic activity and tyrosine kinase inhibitory effects of 1-benzyl-3-(1H-indol-3-ylmethylidene)-1,3-dihydro-2H-indol-2-one derivatives, also indications for such compounds.

### SUMMARY OF THE INVENTION

In an aspect, the present invention provides a compound having the following formulation for use in the treatment of human breast, lung, pancreas, colon and/or kidney cancer:

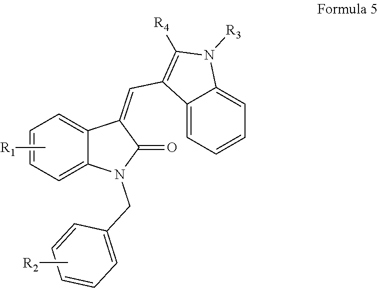

wherein;
   R₁ is a hydrogen, alkyl, methoxy, trifluoromethoxy, halogen, nitro, cyano, carboxamide, sulfonic acid, sodium sulfonate or sulfonamide group;
   R₂ is a hydrogen, alkyl, trifluoromethyl, methoxy, trifluoromethoxy, halogen, nitro, amine, dimethylamine, cyano, carboxylic acid, sulfonic acid or hydroxyl group; and
   R₃ and R₄ are independently of each other a hydrogen or methyl group.

In embodiments of the present invention, the R₁, R₂, R₃ and R₄ groups are selected from the following groups:

| | Formula 5 |
|---|---|
| R₁ | H, 5-CH₃, 5-CH(CH₃)₂, 5-C(CH₃)₃, 5-OCH₃, 5-OCF₃, 5-F, 5-Cl, 5-Br, 5-I, 5-NH₂, 5-NO₂, 5-CN, |
| | 5-COOH, 5-CONH₂, 5-SO₃H, 5-SO₃Na, 5-SO₂NH₂, 7-CH₃, 7-CF₃, 7-OCH₃, 7-F, 7-Cl, 7-Br, 5,7- |
| | diCH₃, 5-F-7-CH₃, 5-Cl-7-CH₃, 7-F-6-Cl, 5,7-diCl, veya 5,7-diBr |
| R₂ | H, 2-CH₃, 2-C₂H₅, 2-CH(CH₃)₂, 2-C(CH₃)₃, 2-CF₃, 2-OCH₃, 2-SCH₃, 2-OCF₃, 2-F, 2-Cl, 2-Br, 2-I, |
| | 2-NO₂, 2-NH₂, 2-CN, 2-COOH, 2-OH, 3-CH₃, 3-C₂H₅, 3-CH(CH₃)₂, 3-C(CH₃)₃, 3-CF₃, 3-OCH₃, |
| | 3-SCH₃, 3-OCF₃, 3-SCF₃, 3-F, 3-Cl, 3-Br, 3-I, 3-NO₂, 3-NH₂, 3-CN, 3-COOH, 3-OH, 4-CH₃, 4- |
| | C₂H₅, 4-CH(CH₃)₃, 4-C(CH₃)₃, 4-CF₃, 4-OCH₃, 4-SCH₃, 4-OCF₃, 4-SCF₃, 4-F, 4-Cl, 4-Br, 4-I, 4- |
| | NO₂, 4-NH₂, 4-N(CH₃)₂, 4-CN, 4-COOH, 4-SO₃H, 4-OH, 2,3-diCH₃, 2,4-diCH₃, 2,5-diCH₃, 2,6- |
| | diCH₃, 3,4-diCH₃, 3,5-diCH₃, 3,5-diCF₃, 2,4-diOCH₃, 2,5-diOCH₃, 3,4-diOCH₃, 3,5-diOCH₃, 2- |
| | CH₃-3-Cl, 2-CH₃-5-Cl, 4-CH₃-3-CF₃, 2-OCH₃-5-CH₃, 2-F-5-CF₃, 4-F-3-CF₃, 2-Cl-5-CF₃, 4-Cl-3- |
| | CF₃, 5-Cl-2-OCH₃, 2,3-diF, 2,4-diF, 2,5-diF, 2,6-diF, 3,4-diF, 3,5-diF, 2,3-diCl, 2,4-diCl, 2,5- |
| | diCl, 2,6-diCl, 3,4-diCl, 3,5-diCl, 2-Cl-4F, 2-Cl-6F veya 4-Br-2-F |
| R₃ | H or CH₃ |
| R₄ | H or CH₃ |

The compound of the present invention is a tyrosine kinase inhibitor besides a compound that stimulates cell apoptosis in cancer.

### BRIEF DESCRIPTION OF THE FIGURES

FIG. 1 shows the effect of Y3 (A) performed for 48 hours and Y8 and Y23 (B) performed for 72 hours on general tyrosine kinase activity in A498 kidney cancer cells.

FIG. 2 shows the effect of Y1, Y2, Y3, Y4, Y5, Y8, Y14 and Y21 on general tyrosine kinase activity of MIA PaCa-2 pancreas cancer cells performed for 72 hours.

FIG. 3 shows the effect of Y4 and 20C on general tyrosine kinase activity of HCT116 colon cancer cells performed for 72 hours.

FIG. 4 shows the analysis comparison of apoptosis values of Y3 performed in A498 kidney cancer cell line compared to controls.

FIG. 5 shows the appropriate dose determination evaluation of Sunitinib malate used for positive control in A498 kidney cancer cell line.

FIG. 6 shows the analysis comparison of apoptosis values of Y1, Y3, Y4, Y5, Y8, Y14 and Sunitinib performed in MiaPaca-2 pancreas cancer cell line-compared controls.

### DETAILED DESCRIPTION OF THE INVENTION

In this detailed description, the cytotoxic activities and tyrosine kinase inhibitory effects of 1-benzyl-3-(1H-indol-3-ylmethylidene)-1,3-dihydro-2H-indol-2-one derivatives, novel 3-arylidene-2-indolinone derivatives of the present invention, are described in detail. It has been determined that the 2-indolinone core can be selected as a pharmacophore group during the development of anticancer agents and that substitutions at positions 1, 3, 5 and 7 of the oxindole ring in these compounds increase antitumor activity.

Based on these findings, the present invention provides novel 1-benzyl-3-(1H-indol-3-ylmethylidene)-1,3-dihydro-2H-indol-2-one derivatives of general formula 5 for use in the treatment of human breast, lung, pancreas, colon and/or kidney cancer. The present invention comprises new derivatized products and methods of synthesis of said new products. The chemical structure of the compounds having Formula 5 is shown below.

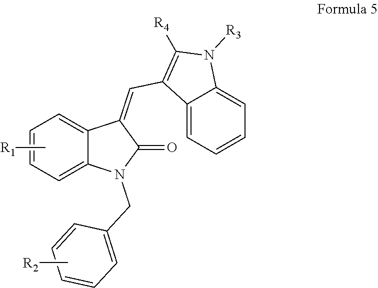

Many 3-arylidene-2-indolinone derivatives have been developed as tyrosine kinase inhibitors effective against kinases which are target proteins in cancer, atherosclerosis, proliferative diseases such as arthritis and metabolic diseases such as diabetes. The new 3-arylidene-2-indolinone derivatives synthesized for this purpose are 1-benzyl-3-(1H-indol-3-ylmethylidene)-1,3-dihydro-2H-indol-2-one derivatives bearing a substituted benzyl residue at the 1 position of the 1H-indol-2,3-dione ring system, different from the existing 3-arylidene derivatives in the literature. These benzyl residue bearing derivatives (Formula 5) have been developed and synthesized.

Thus, in an aspect, the present invention provides novel 1-benzyl-3-(1H-indol-3-ylmethylidene)-1,3-dihydro-2H-indol-2-one derivatives having the general formula 5:

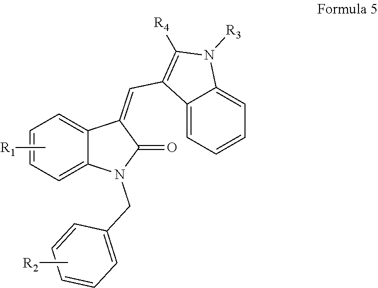

wherein R₁, Rz, R₃ and R₄ can be suitably selected as one of the following:
   R₁═H, 5-CH₃, 5-CH(CH₃)₂, 5-C(CH₃)₃, 5-OCH₃, 5-OCF₃, 5-F, 5-Cl, 5-Br, 5-I, 5-NH₂, 5-NO₂, 5-CN, 5-COOH, 5-CONH₂, 5-SO₃H, 5-SO₃Na, 5-SO₂NH₂, 7-CH₃, 7-CF₃, 7-OCH₃, 7-F, 7-Cl, 7-Br, 5,7-diCH₃, 5-F-7-CH₃, 5-Cl-7-CH₃, 7-F-6-Cl, 5,7-diCl, 5,7-diBr,
   R₂═H, 2-CH₃, 2-C₂H₅, 2-CH(CH₃)₂, 2-C(CH₃)₃, 2-CF₃, 2-OCH₃, 2-SCH₃, 2-OCF₃, 2-F, 2-Cl, 2-Br, 2-I, 2-NO₂, 2-NH₂, 2-CN, 2-COOH, 2-OH, 3-CH₃, 3-C₂H₅, 3-CH(CH₃)₂, 3-C(CH₃)₃, 3-CF₃, 3-OCH₃, 3-SCH₃, 3-OCF₃, 3-SCF₃, 3-F, 3-Cl, 3-Br, 3-I, 3-NO₂, 3-NH₂, 3-CN, 3-COOH, 3-OH, 4-CH₃, 4-C₂H₅, 4-CH(CH₃)₃, 4-C(CH₃)₃, 4-CF₃, 4-OCH₃, 4-SCH₃, 4-OCF₃, 4-SCF₃, 4-F, 4-Cl, 4-Br, 4-I, 4-NO₂, 4-NH₂, 4-N(CH₃)₂, 4-CN, 4-COOH, 4-SO₃H, 4-OH, 2,3-diCH₃, 2,4-diCH₃, 2,5-diCH₃, 2,6-diCH₃, 3,4-diCH₃, 3,5-diCH₃, 3,5-diCF₃, 2,4-diOCH₃, 2,5-diOCH₃, 3,4-diOCH₃, 3,5-diOCH₃, 2-CH₃-3-Cl, 2-CH₃-5-Cl, 4-CH₃-3-CF₃, 2-OCH₃-5-CH₃, 2-F-5-CF₃, 4-F-3-CF₃, 2-Cl-5-CF₃, 4-Cl-3-CF₃, 5-Cl-2-OCH₃, 2,3-diF, 2,4-diF, 2,5-diF, 2,6-diF, 3,4-diF, 3,5-diF, 2,3-diCl, 2,4-diCl, 2,5-diCl, 2,6-diCl, 3,4-diCl, 3,5-diCl, 2-Cl-4F, 2-Cl-6F, 4-Br-2-F,

$${R}_{3} = H , {CH}_{3} ,$$

$${R}_{4} = H , {CH}_{3} .$$

In another aspect, the present invention provides a method for the synthesis of the compounds of the invention. Derivatives of 1-benzyl-1H-indole-2,3-dione (2) are obtained from the reaction of 1H-indole-2,3-dione (1) derivatives in the presence of dimethylformamide (DMF), catalyzed by K₂CO₃ and benzyl halide. For example, the benzyl halide can preferably be selected from a group comprising benzyl iodide, benzyl bromide and benzyl chloride. More preferably, it is benzyl chloride (Torres, JC, Pinto, A. C., Garden, S. J. (2004). “Application of a catalytic palladium biaryl synthesis reaction, via C—H functionalization, to the total synthesis of Amaryllidaceae alkaloids” Tetrahedron, 60(44), 9889-9900; Vine, K. L., Locke, J. M., Ranson, M., Pyne, S. G., Bremner, J. B. (2007). “An investigation into the cytotoxicity and mode of action of some novel N-alkyl-substituted isatins”, Journal of Medicinal Chemistry, 50(21), 5109-5117). Secondly, 1,3-dihydro-2H-indol-2-one derivatives (3) are obtained from the reaction of 1,3-dihydro-2H-indol-2,3-dione (2) and hydrazine hydrate. In the third step, 1-benzyl-3-(1H-indol-3-ylmethylidene)-1,3-dihydro-2H-indol-2-one derivatives (5) are synthesized by reacting 1,3-dihydro-2H-indol-2-one derivatives (3) with 1H-indole-3-carbaldehydes (4) in ethanol and piperidine medium (Scheme 1) (Mologni, L., Rostagno, R., Brussolo, S., Knowles, P. P., Kjaer, S., Murray-Rust, J, Rosso, E., Zambon, A., Scapozza, L., McDonald, N. Q., Lucchini, V., Gambacorti-Passerini, C. (2010). “Synthesis, structure-activity relationship and crystallographic studies of3-substituted indolin-2-one RET inhibitors”, Bioorganic & Medicinal Chemistry, 18, 1482-1496; Zhong, Y., Xue, M., Zhao, X., Yuan, J, Liu, X., Huang, J., Zhao, Z., Li, H., Xu, Y. (2013). Substituted indolin-2-ones as p90 ribosomal S6 protein kinase 2 (RSK2) inhibitors: Molecular docking simulation and structure-activity relationship analysis. Bioorganic & Medicinal Chemistry, 21, 1724-1734). The analytical purity of compounds (5) has been determined by elemental analysis and their structures have been confirmed by spectral analysis (IR, 1H NMR, 13C NMR-APT, HSQC-2D, HMBC-2D and LCMS).

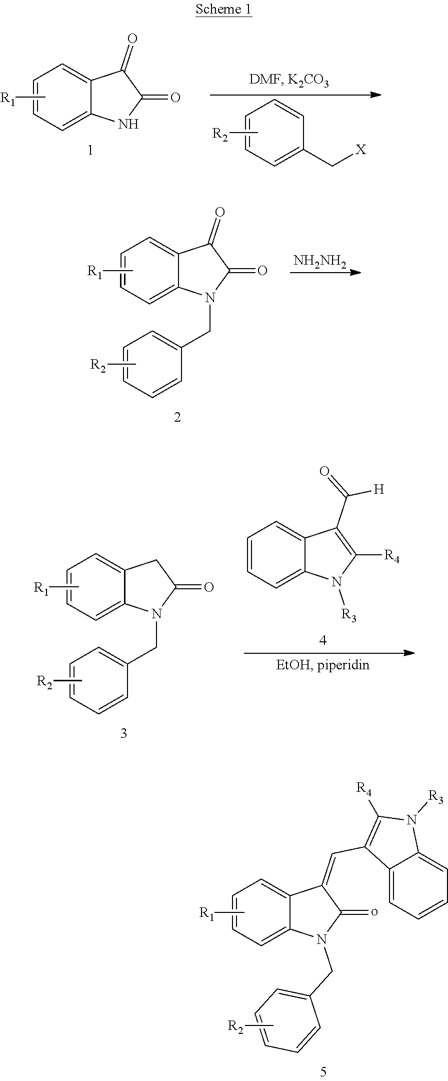

For the compounds (1, 2, 3, 4, 5) given in Scheme 1, R₁, Rz, R₃ and R₄ are suitably selected as follows:
R₁ is hydrogen, alkyl, methoxy, trifluoromethoxy, halogen, nitro, cyano, carboxamide, sulfonic acid, sodium sulfonate, sulfonamide.
R₂ is hydrogen, alkyl, trifluoromethyl, methoxy, trifluoromethoxy, halogen, nitro, amine, dimethylamine, cyanocarboxylic acid, sulfonic acid, hydroxyl.
R₃ and R₄ are hydrogen and methyl.

The synthesis and purification steps of the compounds of the present invention are detailed in the following examples.

### EXAMPLES

### Cellular Assays and Methods

The cytotoxicity, general tyrosine kinase inhibitory effects and cell death (apoptosis/necrosis) assays of 1-benzyl-3-(1H-indol-3-ylmethylidene)-1,3-dihydro-2H-indol-2-one derivatives (Formula 5) of the present invention are studied by the following methods. The cancer cells tested have been human kidney (CAKI-1, A498), pancreas (MIA PaCa-2), colon (HT-29 AND HCT-116), breast (MCF-7 and MDA-MB-231) and lung (A549) cancer cells. HUVEC (human umbilical vein endothelial cell), MCF-10F (human breast epithelial cell) and Beas2b (human lung bronchial epithelial cell) cell lines have been used as healthy control cells in MTT experiments.

### Cell Culture Treatments

Human kidney (A498), lung (A549), pancreas (Miapaca-2), colon (HT29 and HCT116) cells are incubated in Dulbecco's Modified Eagle's Medium (DMEM) containing 10% fetal bovine serum, 100 IU/mL penicillin, 100 g/mL streptomycin and 2 mmol/L glutamine, human breast (MCF-7 and MDAMB-231) cells in DMEM: F12, and human CAKI-1 renal carcinoma cells in McCoy's 5A medium are incubated under standard culture conditions (37° C., humidified environment containing 5% CO2). Beas2b (human lung bronchiole epithelial cell) and HEK293 (renal tubule epithelium) cells used as normal cell lines were cultured in RPMI medium, and HUVEC (human umbilical cord endothelial cells) and MCF-10F (human breast epithelial cell) cells are cultured in DMEM medium containing 100 g/ml penicillin/streptomycin. The cells, which covered 70% of the culture flask, are washed with phosphate buffer and then removed from the surface of the flask by application of trypsin-ethylenediamine tetraacetic acid (Trypsin-EDTA; 0.25%) solution, passaged, counted and seeded on appropriate plates before each experiment. Cells are treated with a total of 45 2-indolinone compounds (5) at concentrations of 0.1, 1, 5 and 10 μM. Stock compounds are prepared in 1 ml dimethylsulfoxide (DMSO) and 4 different dilutions were prepared with medium and performed to the cells. Control group cells are treated with DMSO at a similar concentration to the experimental groups. The cells are collected at 24, 48 and 72 hours following the application of 2-indolinone compounds (5) with IC₅₀ values below, and then cytotoxicity, general tyrosine kinase activity determination and cell death (apoptosis/necrosis) assays are performed on cell lysates, respectively.

### Assessment of Cell Viability by MTT Assay

MTT (3-(4,5-dimethylthiazol-2-yl)-2,5-diphenyl-2H-tetrazolium bromide) colorimetric assay provides quantitation of viable cells with ongoing mitochondrial activity in culture medium. According to the standard procedure in cell culture conditions, cells are maintained in RPMI-1640 and DMEM culture media containing 10% FBS and 1% penicillin/streptomycin. The trypan blue test is used for cell counts.

The effects of 45 2-indolinone compounds (5) on cell viability in all cancer cell lines are investigated by MTT assay. Cells are seeded in a 96-well flat bottom plate with 5000 cells in each well and 2-indolinone compounds (5) at concentrations of 0.1, 1, 5 and 10 μM are performed to the cells the next day. After 48 and/or 72 hours following treatment with 2-indolinone compounds, 50 μl of the supernatant is slowly discarded (as it was acid medium) and MTT at a concentration of 5 mg/ml is added to each well in a volume of 10 μl. After incubation for 4 hours, the formazan crystals formed in the wells are dissolved by adding 100 μl DMSO and the optical density is read at 570 nm test wavelength and 630 nm reference wavelength. Cell viability is expressed as % cell viability by comparing the absorbance of control and test wells. IC₅₀ value is calculated from the dose-response curves prepared for each compound. In addition, the possible DMSO effect is eliminated by preparing negative control wells compatible with the amount of DMSO contained in the compounds. To understand the reaction of the compounds with MTT, cell-free (MTT+compounds) negative controls are prepared at the concentrations used (Mosmann, 1983). Since the 2-indolinone compounds (5) are selected to kill cancer cells at IC₅₀ values below 1 μM concentration, these substances are first tested on healthy cells and those that did not cause toxic effects on healthy cells (did not reduce cell viability below 80%) are determined. The absorbance values obtained are compared to the control and statistically evaluated by unpaired t-test using “Graphpad Prism 5.04” software. “P<0.05” is accepted as significant. When evaluating cytotoxic effects, the present inventors aim for the inventive compounds to be effective in cancer cells and ineffective in healthy cells. The inventive compounds have also been compared with sunitinib, one of the reference chemotherapy drugs currently used in treatment.

### Evaluation of General Tyrosine Kinase Inhibitor (TKI) Effect (ELISA)

Takara Universal Tyrosine Kinase Assay Kit is used to determine the effects of 11 2-indolinone compounds (Y1, Y2, Y3, Y4, Y5, Y8, Y14, Y14, Y21, Y23, 20C, 22C) on general tyrosine kinase activity in cancer cells. Cells are seeded in 6-well plates (4.10⁵ cells/well) and the IC₅₀ concentration of each selected molecule is performed to the cells for 48 or 72 hours. Cells in the control group are incubated with medium only. At the end of the incubation period, the cells in each well are washed with PBS and cells are removed from the wells using a cell scraper via 1 ml of the extraction buffer in the kit. The cell lysate is transferred to ependorf tubes and centrifuged at 10.000 g for 10 minutes at +4° C. and the supernatant is used as a sample for activity determination. After the extracts obtained from the control and experimental groups are diluted 1:25, 40 μl extracts is transferred to two wells in the 96-well plate in the kit. In addition, the standard solution in the kit is diluted according to the kit instructions and each concentration is transferred 40 μl into three wells. 10 μl of 40 mM ATP-2Na solution is added to all wells and the plate is incubated at 37° C. for 30 minutes. After incubation, all wells are washed 4 times with wash buffer. 100 μl of blocking solution is added to each well and incubated again at 37° C. for 30 minutes. After incubation, the liquid in the wells is discarded and 50 μl of anti-phosphotyrosine-HRP solution is added to each well and incubated at 37° C. for 30 minutes. The wells are washed again four times with wash buffer and 100 μl of HRP-substrate solution (TMBZ) is added to each well and incubated at 37° C. for 15 minutes. The reaction is finished by adding 100 μl of stop solution to each well and the optical density in each well is measured at 405 nm with a microplate reader (BioTek, USA). The tyrosine kinase activity in each sample is calculated from the standard curve obtained from the absorbance values of the standard solutions with the kit (Chan W Y, Lau P M, Yeung K W, Kong S K. The second generation tyrosine kinase inhibitor dasatinib induced eryptosis in human erythrocytes—An in vitro study. Toxicol Lett. 2018; 295:10-21). This activity is normalized compared to the control group by dividing by the amount of protein in each sample.

### Apoptosis/Necrosis Detection (Flow Cytometry)

Whether 2-indolinone derivatives (5) with IC₅₀ values below 1 μM, the highest selectivity index and general TKI activity stimulate apoptosis and necrosis are determined by Annexin V and propidium iodine staining in pancreas and kidney cancer cells. After incubation with 2-indolinone compounds (IC₅₀ μM concentration; 48 and/or 72 hours), cells are washed twice with PBS and the cell pellet is suspended in Annexin V binding buffer. After this incubation, cells are incubated with FITC-conjugated Annexin V and propidium iodine staining solution for 15 minutes in the dark and at room temperature. Then, cells are analyzed by flow cytometry (Beckman coulter, Navios) (Wlodkowic, D., Skommer, J, Darzynkiewicz, Z. 2009. Flow cytometry-based apoptosis detection, Methods Mol Biol., 559, 1-14).

### Findings of Cell Viability Tests

Cell viability is significantly reduced at 48 and 72 hours by rarely 5 μM and frequently 10 μM of the compounds of the present invention. IC₅₀ values are calculated at 24, 48 and 72 hours after treatment in human breast, lung and kidney cancer cells treated with the compounds with the most effective cytotoxic dose (IC₅₀<1 μM). Among the compounds of the present invention, those that killed cancer cells by at least 50% (p<0.05) are selected and tested whether a 10-fold concentration of the M concentration at the IC₅₀ value is toxic to normal healthy cells. The in vitro results obtained are shown in Table 1.

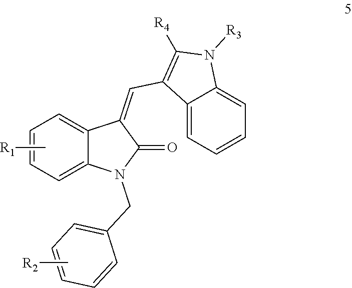

TABLE 1
IC₅₀ values (uM) of compounds with general formula 5 and sunitinib malate on human
breast cells (MCF-7 and MDA-MB-231), lung (A549), kidney (CAKI-1 and A498) cancer cells and
normal cell lines (MCF-10F/Beas2b and HUVEC).

| | | R | | | | Breast | | Lung | Kidney | | Healthy Cells | | Selectivite Index (SI) | |
|---|---|---|---|---|---|---|---|---|---|---|---|---|---|---|
| | Comp. | R₁ | R₂ | R₃ | R₄ | MCF-7 | MDA-MB-231 | A549 | CAK3-1 | A49B | MCF-10F/Beas2b | HUVEC | HUVEC | MCF-10F/Beas2b |
| 1 | Y14 | 5- | H | CH₃ | H | 6.09 | > 10 | > 10 | > 10 | > 10 | 6.76 | > 10 | | |
| | | OCF₃ | | | | | | | | | | | | |
| 2 | 22C | 7-Cl | H | H | H | 0.57 | 8.40 | > 10 | 2.49 | > 10 | > 10 | > 10 | > 17.54 | > 17.54 |
| 3 | 28C | 7-Cl | 4- | H | H | > 10 | > 10 | > 10 | 7.66* | 0.6* | 1.78* | > 10 | > 15.6 | 1.5 |
| | | | CH₃ | | | | | | | | | | | |
| A | 47C | 5- | 4-Cl | H | H | 5.00 | > 10 | 2.65 | 4.66 | 0.9* | 2.6* | > 10 | > 11.11 | 2.88 |
| | | CH₃ | | | | | | | | | | | | |
| 5 | Y3 | 5- | H | H | H | 4.75 | 3.57 | 3.97 | 4.01 | 0.87 | > 10 | 1.65 | 1.89 | > 11.49 |
| | | CH₃ | | | | | | | | | | | | |
| 6 | Y5 | 5-F | H | H | H | 4.44 | 7.94 | 7.81 | 3.42 | 4.32 | > 10 | 2.17 | | |
| 7 | 5C | 5- | 4- | H | H | 3.84* | 5.8* | 2.96 | 6.98* | 2.26* | 3.46 | 2.72 | | |
| | | OCF₃ | Cl | | | | | | | | | | | |
| 8 | 35C | 7-F | 4- | H | H | > 10 | > 10 | 5.93 | 4.50 | 3.09* | 3.23 | 1.62 | | |
| | | | CH₃ | | | | | | | | | | | |
| g | Y1 | H | H | H | H | 6.34 | 9.82 | 9.72 | 3.38 | 4.11 | > 10 | 2.84 | | |
| 10 | Y21 | H | 4- | H | H | 8.77 | > 10 | > 10 | > 10 | 4.06 | > 10 | 2.46 | | |
| | | | CH₃ | | | | | | | | | | | |
| 11 | Y2 | 5-Cl | H | H | H | 4.16 | 4.96 | 4.04 | 4.25 | 2.36 | > 10 | 1.06 | | |
| 12 | Y4 | 5- | H | H | H | > 10 | > 10 | > 10 | 3.79 | 4.23 | > 10 | 2.44 | | |
| | | OCF₃ | | | | | | | | | | | | |
| 13 | Y8 | 5- | 3-F | H | H | 5.79 | 7.36 | 8.76 | 3.28 | 0.93 | > 10 | 2.20 | 2.36 | > 10.75 |
| | | CHa | | | | | | | | | | | | |
| 14 | Y23 | 5-Cl | H | CHS | H | 4.90 | 4.72 | > 10 | 4.98 | 0.89 | > 10 | 4.18 | 4.70 | > 11.23 |
| 15 | 51C | H | 3-F | H | H | > 10 | > 10 | > 10 | > 10 | 6.22 | > 10 | 5.70 | | |
| 16 | 20C | 5-Cl | 3- | H | H | > 10 | > 10 | 4.04 | 6.19* | 0.97* | 3.68* | 1.52 | | |
| | | | CH₃ | | | | | | | | | | | |
| 17 | 27C | 7-Cl | 3- | H | H | 3.67 | 0.81 | > 10 | >10 | 2.40 | >10 | 1.04* | 1.28 | > 12.34 |
| | | | CH₃ | | | | | | | | | | | |
| 18 | 31C | 7-F | 4-F | H | H | 7.1 | 9.09* | 3.64 | 9.14* | 2.85 | 4.42 | 1.26 | | |
| 19 | 50C | H | 3- | H | H | 4.15* | 4.3 | 4.60 | 4.96 | 3.85* | 4.37 | 0.86 | | |
| | | | CH₃ | | | | | | | | | | | |
| 20 | 45C | 5- | 4-F | H | H | >10 | > 10 | 2.22 | 4.91* | 0.78* | 2.89* | 2.62 | | |
| | | CH₃ | | | | | | | | | | | | |
| 21 | 36C | 5,7- | H | H | H | >10 | > 10 | 2.35 | 4.01* | 1.46* | 3.6 * | 2.24 | | |
| | | diCl | | | | | | | | | | | | |
| 22 | 3C | 5- | 4-F | H | H | 5.06 | 2.27 | 4.22 | 3.70 | 7.53* | 2.98 | 4.30 | | |
| | | OCF₃ | | | | | | | | | | | | |
| 23 | 19C | 5-Cl | 4-Cl | H | H | 3.16 | > 10 | 5.55 | > 10 | 0.51 | 1.87 | 1.87 | | |
| 24 | Y22 | 5-Cl | 4- | H | H | > 10 | > 10 | > 10 | > 10 | > 10 | 1.1 | 2.34 | | |
| | | | CH₃ | | | | | | | | | | | |
| 25 | 48C | 5- | 3- | H | H | 3.96* | > 10 | 3.00 | 3.46* | 0.73* | 3.35* | 1.07 | | |
| | | CH₃ | CH₃ | | | | | | | | | | | |
| 26 | 34C | 7-F | 3- | H | H | > 10 | > 10 | 4.70 | 7.61 | 3.64 | 4.10 | 1.69 | | |
| | | | CH₃ | | | | | | | | | | | |
| 27 | Y13 | 5- | H | CH₃ | H | > 10 | > 10 | > 10 | >10 | >10 | >10 | 3.37 | | |
| | | CHa | | | | | | | | | | | | |
| 28 | 12C | 5-F | 4Cl | H | H | 2.63* | 6.80* | 1.68 | 2.81 | 0.63* | 2.46* | 0.55 | | |
| 29 | 17C | 5-Cl | 4-F | H | H | 4.76* | > 10 | 1.11 | 4.62* | 0.77* | 2.66* | 1.24 | | |
| 30 | 24C | 7-Cl | 4-F | H | H | 5.08 | 7.41 | 8.08 | 5.72* | 2.60 | 1.26 | 1.26 | | |
| 31 | Y12 | H | H | CH₃ | H | > 10 | > 10 | > 10 | > 10 | > 10 | > 10 | > 10 | | |
| 32 | 6C | 5- | 3- | H | H | 4.72 | 9.87 | > 10 | 9.66 | > 10 | > 10 | > 10 | | |
| | | OCFa | CH₃ | | | | | | | | | | | |
| 33 | 11C | 5-F | 3-Cl | H | H | >10 | 7.25 | > 10 | 9.81 | > 10 | > 10 | > 10 | | |
| 34 | 23C | 7-Cl | 3-F | H | H | 3.07 | > 10 | > 10 | 4.88 | 8.33 | > 10 | > 10 | | |
| 35 | 26C | 7-Cl | 4-Cl | H | H | 3.55 | > 10 | > 10 | 4.50 | 6.92 | > 10 | > 10 | | |
| 36 | 10C | 5-F | 4-F | H | H | > 10 | 8.13* | 3.49 | 6.69* | 3.81 | 2.99 | 4.65 | | |
| 37 | 13C | 5-F | 3- | H | H | > 10 | > 10 | 3.43 | > 10 | 2.66 | 2.63 | 2.24 | | |
| | | | CH₃ | | | | | | | | | | | |
| 38 | 2C | 5- | 3-F | H | H | 8.82 | 2.91 | 3.58 | 5.46* | 4.78 | 3.07 | 2.56 | | |
| | | OCF₃ | | | | | | | | | | | | |
| 39 | Y25 | 5-F | 4- | H | H | 4.83* | 3.40 | 3.31 | 7.00* | 3.15 | 2.72 | 0.13 | | |
| | | | CH₃ | | | | | | | | | | | |
| 40 | 29C | 7-F | H | H | H | 4.35 | > 10 | > 10 | 4.12 | > 10 | > 10 | 0.95* | | |
| 41 | 30C | 7-F | 3-F | H | H | 4.31 | 4.21 | 6.80 | 7.70* | 6.57 | 1.9 | 0.94* | | |
| 42 | 25C | 7-Cl | 3-Cl | H | H | 4.50 | > 10 | > 10 | 8.07* | 2.91 | 1.81 | 1.81 | | |
| 43 | 18C | 5-Cl | 3-Cl | H | H | 5.53 | 7.99 | 6.57 | 3.46 | 1.75 | 1.07 | 1.07 | | |
| 44 | 32C | 7-F | 3-Cl | H | H | 3.86 | 4.53 | > 10 | 8.76* | 8.26 | 1.35 | 1.00* | | |
| 45 | 4C | 5- | 3-Cl | H | H | 3.06* | 3.07* | 2.42 | 2.79* | 3.05 | 1.73 | 0.97 | | |
| | | OCF₃ | | | | | | | | | | | | |
| | Sunitinib | | | | | 9.78 | > 10 | > 10 | 5.13 | 3.71 | > 10 | 2.94 | | |
| | malate | | | | | | | | | 3.46* | | | | |

* Effective 72 hours after incubation.

When evaluating cytotoxic effects, the present inventors aim for the inventive compounds to be effective in cancer cells and ineffective in healthy cells. The inventive compounds have also been compared with sunitinib, one of the reference chemotherapy drugs currently used in treatment

TABLE 2
IC₅₀ values (uM) of compounds with general formula 5 and sunitinib malate on human
pancreas (MIAPaCa-2) and colon (HT29, HCT-116) cancer cells and normal cell lines (MCF-
10F/Beas2b and HUVEC).

| | | R | | | | COLON | | PANCREAS | Healthy Cells | | Selectivite Index | |
|---|---|---|---|---|---|---|---|---|---|---|---|---|
| | Comp. | R₁ | R₂ | R₃ | R₄ | HT-29 | HCT-116 | MIAPaCa-2 | MCF-10F/Beas2b | HUVEC | MCF-10F/Beas2b | HUVEC |
| 1 | Y14 | 5-OCF₃ | H | CH₃ | H | > 10 | > 10 | 0.15 | 6.76 | > 10 | 45.06 | > 66.66 |
| 2 | 22C | 7-Cl | H | H | H | > 10 | > 10 | > 10 | > 10 | > 10 | | |
| 3 | 28C | 7-Cl | 4-CH₃ | H | H | > 10 | > 10 | > 10 | 1.78* | > 10 | | |
| 4 | 47C | 5-CH₃ | 4-CI | H | H | > 10 | > 10 | > 10 | 2.6* | > 10 | | |
| 5 | Y3 | 5-CH₃ | H | H | H | 2.43 | 2.05 | 0.13 | > 10 | 1.65 | > 76.92 | 12.69 |
| 6 | Y5 | 5-F | H | H | H | 2.98 | 2.05 | 0.27 | > 10 | 2.17 | > 37.04 | 8.04 |
| 7 | 5C | 5-OCF₃ | 4-Cl | H | H | 0.44 | 1.97 | > 10 | 3.46 | 2.72 | 7.86 | 6.18 |
| 8 | 35C | 7-F | 4-CH₃ | H | H | 1.01 | 0.23 | 0.62 | 3.23 | 1.62 | 14.04 | 7.04 |
| | | | | | | | | | | | 5.20 | 2.61 |
| 9 | Y1 | H | H | H | H | 1.81 | 2.64 | 0.43 | > 10 | 2.84 | > 23.25 | 6.60 |
| 10 | Y21 | H | 4-CH₃ | H | H | > 10 | 3.14 | 0.40 | > 10 | 2.46 | > 25.00 | 6.15 |
| 11 | Y2 | 5-Cl | H | H | H | 1.52 | 1.44 | 0.48 | > 10 | 1.06 | > 20.83 | 2.21 |
| 12 | Y4 | 5-OCF₃ | H | H | H | 1.75 | 0.57 | 0.53 | > 10 | 2.44 | > 17.54 | 4.28 |
| | | | | | | | | | | | > 18.87 | 4.60 |
| 13 | Y8 | 5-CH₃ | 3-F | H | H | 5 | 1.21 | 0.54 | > 10 | 2.20 | > 18.52 | 4.07 |
| 14 | Y23 | 5-Cl | H | CH₃ | H | 4.18 | 1.62 | > 10 | > 10 | 4.18 | | |
| 15 | 51C | H | 3-F | H | H | 0.93 | 1.13 | 5.20 | > 10 | 5.70 | > 10.75 | 6.13 |
| 16 | 20C | 5-Cl | 3-CH₃ | H | H | 1.16 | 0.27 | 1.39 | 3.68* | 1,.52 | 13.63 | 5.63 |
| 17 | 27C | 7-Cl | 3-CH₃ | H | H | > 10 | > 10 | > 10 | > 10 | 1.04* | | |
| 18 | 31C | 7-F | 4-F | H | H | 3.95 | 0.39 | 0.73 | 4.42 | 1.26 | 11.33 | 3.23 |
| | | | | | | | | | | | 6.05 | 1.72 |
| 19 | 50C | H | 3-CH₃ | H | H | 1.49 | 0.50 | 2.96 | 4.37 | 0.86 | 8.74 | 1.72 |
| 20 | 45C | 5-CH₃ | 4-F | H | H | > 10 | 0.47 | > 10 | 2.89* | 2.62 | 6.14 | 5.57 |
| 21 | 36C | 5.7-diCI | H | H | H | > 10 | 2.51 | 0.46 | 3.6 * | 2.24 | 7.83 | 4.87 |
| 22 | 3C | 5-OCF₃ | 4-F | H | H | 1.38 | 5.74 | 0.95* | 2.98 | 4.30 | | |
| 23 | 19C | 5-Cl | 4-Cl | H | H | > 10 | > 10 | > 10 | 1.87 | 1.87 | | |
| 24 | Y22 | 5-Cl | 4-CH₃ | H | H | > 10 | 2.34 | 0.49 | 1.1 | 2.34 | | |
| 25 | 48C | 5-CH₃ | 3-CH₃ | H | H | 1.46 | 0.60 | 2.02 | 3.35 | 1.07 | | |
| 26 | 34C | 7-F | 3-CHa | H | H | > 10 | 0.64 | > 10 | 4.10 | 1.69 | | |
| 27 | Y13 | 5-CH₃ | H | CH₃ | H | 3.37 | 4.58 | 2.2 | >10 | 3.37 | | |
| 28 | 12C | 5-F | 4Cl | H | H | 1.08 | 0.64 | 0.49 | 2.46 | 0.55 | | |
| 29 | 17C | 5-Cl | 4-F | H | H | 2.07 | 0.93 | 1.32 | 2.66 | 1.24 | | |
| 30 | 24C | 7-Cl | 4-F | H | H | 0.73 | 2.35 | 1.12 | 1.26 | 1.26 | | |
| 31 | Y12 | H | H | CH₃ | H | > 10 | 1.46 | > 10 | > 10 | > 10 | | |
| 32 | 6C | 5-OCF₃ | 3-CH₃ | H | H | > 10 | > 10 | > 10* | > 10 | > 10 | | |
| 33 | 11C | 5-F | 3-Cl | H | H | > 10 | > 10 | > 10* | > 10 | > 10 | | |
| 34 | 23C | 7-Cl | 3-F | H | H | > 10 | > 10 | > 10* | > 10 | > 10 | | |
| 35 | 26C | 7-Cl | 4-Cl | H | H | > 10 | > 10 | > 10* | > 10 | > 10 | | |
| 36 | 10C | 5-F | 4-F | H | H | 2.29 | 3.96 | 2.77 | 2.99 | 4.65 | | |
| 37 | 13C | 5-F | 3-CH₃ | H | H | > 10 | > 10 | 1.51* | 2.63 | 2.24 | | |
| 38 | 2C | 5-OCF₃ | 3-F | H | H | > 10 | 2.88 | 7.35* | 3.07 | 2.56 | | |
| 39 | Y25 | 5-F | 4-CH₃ | H | H | > 10 | 1.46 | 1.14 | 2.72 | 0.13 | | |
| 40 | 29C | 7-F | H | H | H | > 10 | > 10 | > 10 | > 10 | 0.95* | | |
| 41 | 30C | 7-F | 3-F | H | H | > 10 | 2.06 | 1.9 | 1.9 | 0.94* | | |
| 42 | 25C | 7-Cl | 3-Cl | H | H | > 10 | 2.90 | 3.23* | 1.81 | 1.81 | | |
| 43 | 18C | 5-Cl | 3-Cl | H | H | 3.12 | 3.94 | 1.54* | 1.07 | 1.07 | | |
| 44 | 32C | 7-F | 3-Cl | H | H | 1.65 | 4.31 | 1.35 | 1.35 | 1.00* | | |
| 45 | 4C | 5-OCF₃ | 3-Cl | H | H | > 10 | 1,13 | 3.67* | 1.73 | 0.97 | | |
| Sunitinib | | | | | | 2.94 | 1.78 | 0.61/4.03* | > 10 | 2.94 | | |
| malate | | | | | | | | 1.18 | | | | |

* Effective 72 hours after incubation.

Cellular assay results show that among the 45 2-indolinone compounds (5) tested, the compounds with IC₅₀ values lower than 1 μM against breast cancer cell lines (MCF-7 and MDA-MB-231) are R₁ is 7-chlorine substituted compound 22C and R₁ is 7-chlorine and R₂ is 3-methyl substituted compound 27C. Compound 22C (IC₅₀=0.57 μM) is found to be the most potent and nontoxic anti-carcinogenic agent for MCF-7 and compound 27C (IC₅₀=0.81 μM) for MDA-MB-231. The selectivity indices (SI) of the two compounds with IC₅₀ values >10 μM in healthy breast cancer MCF-10F cell lines are >17 and >12, respectively. These data indicate that compounds 22C and 27C are much more potent and have higher SI values than the reference compound sunitinib malate (IC50=9.78 μM for MCF-7; >10 μM for MDA-MB-231) (Table 1).

Among the compounds tested, R₁ is 5-chlorine and R₂ is 4-methyl substituted compound 17C (IC₅₀=1.11 μM) is found to be the most effective compound against lung cancer A549 cell line. However, the cytotoxicity of this substance is found to be high in healthy cells. These findings suggest that compound 17C and most of the tested compounds are more potent than sunitinib malate (IC₅₀=>10 μM) (Table 1).

None of the compounds tested against the kidney cancer CAKI-1 cell line had IC₅₀ values lower than 1 μM. Most of the tested compounds showed cytotoxicity to CAKI-1 cell lines in the range of 2.49-9.81 μM. However, ten compounds showed cytotoxicity against kidney cancer A498 cell line at concentrations lower than 1 μM. R₁ is 5-chlorine and R₂ is 4-chlorine substituted compound 19C (IC₅₀=0.51 μM), R₁ is 7-chlorine and R₂ is 4-methyl substituted compound 28C (IC₅₀=0.6 μM), R₁ is 5-fluorine and R₂ is 4-chlorine substituted compound 12C (IC₅₀=0.63 μM), R₁ is 5-methyl and R₂ is 3-methyl substituted compound 48C (IC₅₀=0.73 μM), and R₁ is 5-chlorine and R₂ is 4-fluorine substituted compound 17C (IC₅₀=0.77 μM), R₁ is 5-methyl and R₂ is 4-fluorine substituted compound 45C (IC₅₀=0.78 μM), R₁ is 5-methyl substituted compound Y3 (IC₅₀=0.87 μM), R₁ is 5-chlorine and R₃ is methyl substituted compound Y23 (IC₅₀=0.89 μM), R₁ is 5-methyl and R₂ is 4-chlorine substituted compound 47C (IC₅₀=0.9 μM) and R₁ is 5-methyl and R₂ is 3-fluorine substituted compound Y8 (IC₅₀=0.93 μM) are the compounds with the strongest cytotoxicity. Among these compounds, Y3, Y8 and Y23 with IC₅₀ values >10 μM in healthy MCF-10F/Beas2b cell lines had selectivity indices >11. The IC₅₀ values of sunitinib malate in CAKI-1 and A498 cell lines are 5.13 and >10 μM, respectively. All effective derivatives within the scope of the present invention exhibited much more potent anticancer activity than sunitinib malate, the reference drug for use in the treatment of renal carcinoma (Table 1).

Fourteen compounds showed cytotoxicity against pancreas MIAPaCa-2 cancer cell line at concentrations lower than 1 μM. R₁ is 5-methyl substituted compound Y3 (IC₅₀=0.13 μM), R₁ is 5-trifluoromethoxy and R₃ is methyl substituted compound Y14 (IC₅₀=0.15 μM), R₁ is 5-fluorine substituted compound Y5 (IC₅₀=0.27 μM), R₂ is4-methyl substituted compound Y21 (IC₅₀=0.40 M), nonsubstituted compound Y1 (IC₅₀=0.43 μM), R₁ is 5,7-dichlorine substituted compound 36C (IC₅₀=0.46 μM), R₁ is 5-chlorine substituted compound Y2 (IC₅₀=0.48 μM), R₁ is 5-chlorine and R₂ is 4-methyl substituted compound Y22 (IC₅₀=0.49 μM), R₁ is 5-fluorine and R₂ is 4-chlorine substituted compound 12C (IC₅₀=0.49 μM), R₁ is 5-trifluoromethoxy substituted compound Y4 (IC₅₀=0.53 μM), R₁ is 5-methyl and R₂ is 3-fluorine substituted compound Y8 (IC₅₀=0.54 μM), R₁ is 7-fluorine and R₂ is 4-fluorine substituted compound 31C (IC₅₀=0.73 μM), R₁ is 7-fluorine and R₂ is 4-methyl substituted compound 35C (IC₅₀=0.62 μM) and R₁ is 5-trifluoromethoxy and R₂ is 4-fluorine substituted compound 3C (IC₅₀=0.95 μM) are the compounds with the strongest cytotoxicity. Among these compounds, the selectivity indices of compounds Y3, Y5, Y1, Y21, Y2, Y4 and Y8 with IC₅₀ values >10 μM in healthy MCF-10F/Beas2b cell lines are >77, 37, 23, 25, 21, 19 and 19, respectively. The findings are indicated that most of these compounds are more selective and potent than sunitinib malate against the MIAPaCa-2 cancer cell line (Table 2).

Cellular assay results showed that among the tested 45 2-indolinone compounds (5), the compounds with IC₅₀ values lower than 1 μM against colon HT-29 cancer cell line are R₁ is 5-trifluoromethoxy R₂ is 4-chlorine substituted compound 5C (IC₅₀=0.44 μM), R₁ is 7-chlorine R₂ is 4-fluorine substituted compound 24C (IC₅₀=0.73 μM) and R₂ is 3-fluorine substituted compound 51C (IC₅₀=0.93 μM). Among these compounds, the selectivity index of compound 51C with IC₅₀ values >10 μM in healthy MCF-10F/Beas2b cell lines is >11. The IC₅₀ value of sunitinib malate against colon HT-29 cancer cell line is determined as 2.94 μM. The data show that compound 51C is more potent and selective than Sunitinib malate against colon HT-29 cancer cell line (Table 2).

Ten compounds showed cytotoxicity against the colon HCT-116 cancer cell line at concentrations lower than 1 μM. R₁ is 7-fluorine and R₂ is 4-methyl substituted compound 35C (IC₅₀=0.23 μM), R₁ is 5-trifluoromethoxy substituted compound Y4 (IC₅₀=0.57 μM), R₁ is 5-chlorine and R₂ is 3-methyl substituted compound 20C (IC₅₀=0.27 μM), R₁ is 7-fluorine and R₂ is 4-fluorine substituted compound 31C (IC₅₀=0.39 μM), R₁ is 5-methyl and R₂ is 4-fluorine substituted compound 45C (IC₅₀=0.47 μM), R₁ is 5-methyl and R₂ is 3-methyl substituted compound 48C (IC₅₀=0.60 μM), R₁ is 7-fluorine and R₂ is 3-methyl substituted compound 34C (IC₅₀=0.64 μM), R₁ is 5-fluorine and R₂ is 4-chlorine substituted compound 12C (IC₅₀=0.64 μM) and R₁ is 5-fluorine substituted compound 17C (IC₅₀=0.93 μM) are found to have the strongest cytotoxicity. Among these compounds, the selectivity index of compound Y4 with IC₅₀ values >10 μM in healthy MCF-10F/Beas2b cell lines is >19. The IC₅₀ value of sunitinib malate against colon HCT-116 cancer cell line is determined as 1.78 μM. The data show that compound Y4 is more potent than Sunitinib malate against colon HCT-116 cancer cell line and SI value is high thereof (Table 2).

The present inventors also demonstrate, with the obtained test results, that these compounds are more potent, less toxic to healthy cells and have higher selectivity indices than sunitinib malate, and that methyl-substituted derivatives are generally less potent than non-substituted derivatives in R₃ (Tables 1 and 2).

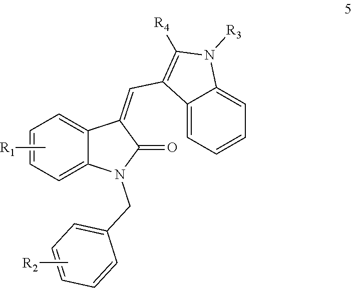

Results of General Tyrosine Kinase Inhibition in A498, Mia-Pa-Ca-2 and HCT-116 Cells Treated with Compounds of Formula 5

The effects of 11 2-indolinone compounds (Y1, Y2, Y3, Y4, Y5, Y8, Y14, Y21, Y23, 20C and 22C) having the structure of formula (5), which are prominent with their cytotoxicity in A498, MIAPaCa-2 and HCT-116 cancer cell lines and nontoxic properties in healthy cells, on general tyrosine kinase activity in cancer cells are determined.

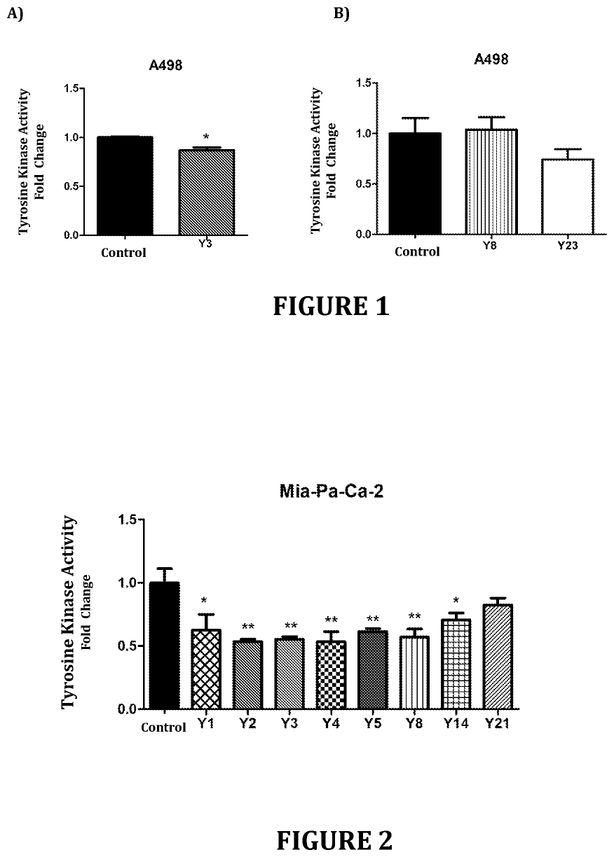

Y3 compound performed to A498 cells at 48 h IC₅₀ concentrations significantly decreased the general tyrosine kinase activity compared to control (p<0.05) (FIG. 1A). Y8 and Y23 compounds performed at 72 h IC₅₀ concentrations did not cause a significant change in general tyrosine kinase activity compared to the control group (p>0.05) (FIG. 1B).

Y1, Y2, Y3, Y4, Y5, Y8, Y14 and Y21 compounds performed at IC₅₀ concentrations to MIAPaCa-2 pancreas cancer cells for 72 hours significantly decreased the general 1 tyrosine kinase activity compared to the control group (*p<0.05; **p<0.01) (FIG. 2).

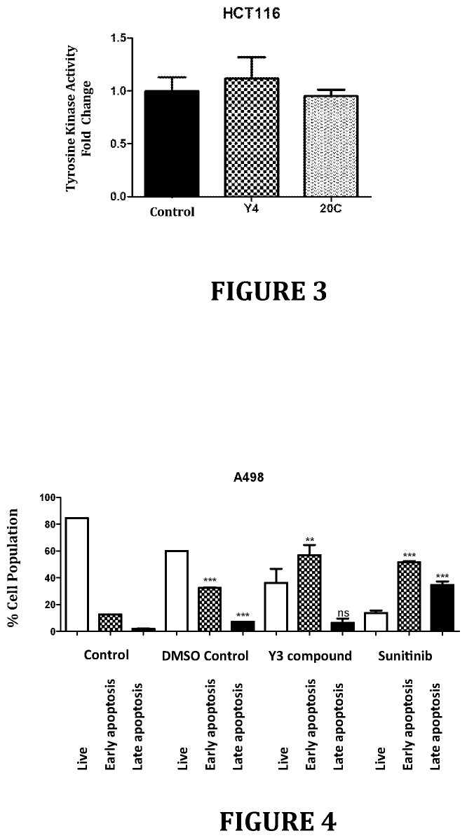

Y4 and 20C compounds performed to HCT-116 colon cancer cells at IC₅₀ concentrations for 72 hours did not cause a significant change in tyrosine kinase activity compared to the control group (p>0.05) (FIG. 3).

In this test, which is performed to determine the 2-indolinone derivative that significantly (at least p<0.05) decreased the general tyrosine kinase activity of the compounds, which are separated from the A series substances performed to kidney, pancreas and colon cancer cells with their nontoxic properties in normal cells according to MTT results, it is determined that Y3 on A498 cells and Y1, Y2, Y3, Y4, Y5, Y8 and Y14 compounds on MIAPaCa-2 cells decreased the general tyrosine kinase activity.

### Results of Apoptosis/Necrosis Determination (Flow Cytometry)

According to MTT results, IC₅₀ values of Y3 performed to A498 cell lines; results of Annexin V/PI flow cytometry apoptosis are statistically significant in terms of viability, early apoptosis and late apoptosis values of compared to control groups. When compared in terms of necrosis, no increase or decrease is observed compared to the control group. Necrosis levels have been at the same values as the control in all experiments (FIG. 4). Therefore, it is not included in the statistical evaluation. Apoptosis rate is increased significantly in the Y3 treated group compared to the control group (FIG. 4). Significance values: **p<0.01 and ***p<0.001.

Treatment with Y3 did not cause necrosis in A498 cells. For these cells, the evaluation is performed in terms of apoptosis.

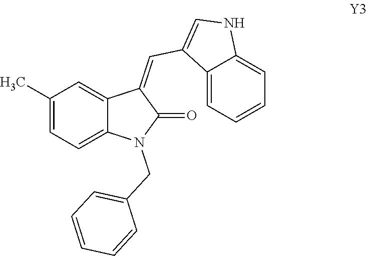 [SMILES: Cc1ccc2c(c1)/C(=C/c1c[nH]c3ccccc13)C(=O)N2Cc1ccccc1]

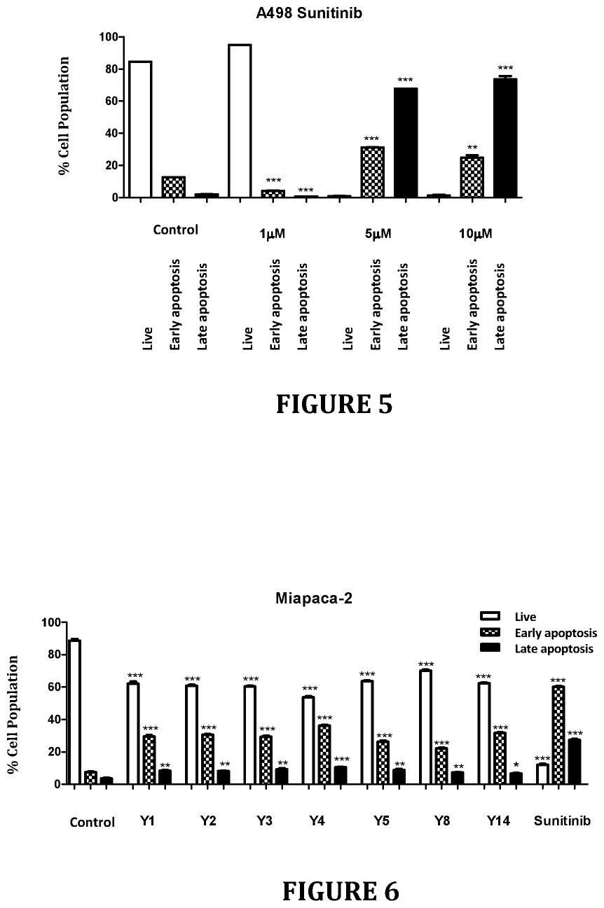

In order to determine the most appropriate dose of Sunitinib malate performed to A498 cell lines, results of Annexin V/PI flow cytometry apoptosis when the viability, early apoptosis and late apoptosis values are compared with the control groups and statistically 10 μM dose is found to be the most significant and this dose is used in the experimental stages (FIG. 6). Significance values: **p<0.01 and ***p<0.001. When compared in terms of necrosis, no increase or decrease is observed compared to the control group. Necrosis levels have been the same as the control in all experiments (FIG. 5).

According to MTT results, Annexin V/PI flow cytometry apoptosis results of Y1, Y3, Y4, Y5, Y8, Y14 and Sunitinib malate performed to MiaPaca-2 cell lines, when the values of early apoptosis and late apoptosis are compared with the control groups and it has been observed that the increase in apoptosis at IC₅₀ values was statistically significant (FIG. 6). Significance values: **p<0.01 and ***p<0.001.

When compared in terms of necrosis, no increase or decrease is observed compared to the control group. Necrosis levels have been the same as the control in all experiments (FIG. 6). Therefore, it is not included in the statistical evaluation. Treatment of Y1, Y3, Y4, Y5, Y8, Y14 and Sunitinib did not cause necrosis in MiaPaca-2 cells. For these cells, the evaluation is performed in terms of apoptosis.

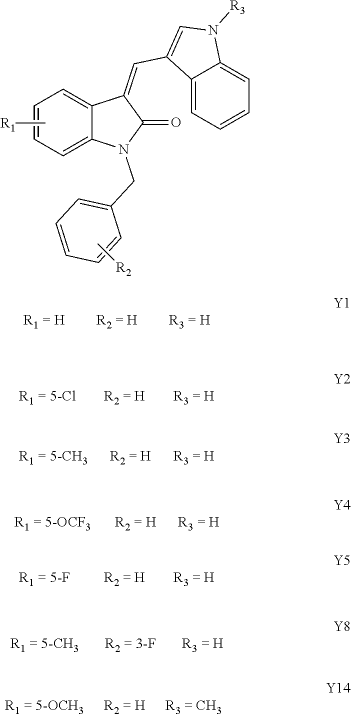

Whether the 2-indolinone derivatives (5) with the highest selectivity index and general TKI activity stimulated apoptosis and necrosis is determined by Annexin V and propidium iodine staining in pancreas and kidney cancer cells. Annexin V/PI flow cytometry apoptosis values of the tested compounds Y1, Y2, Y3, Y4, Y8 and Y14 are found to be 40% and above in correlation with MTT results. The apoptosis rates of the control groups and the substances are analyzed by t test in GraphPad version 9.0.

The apoptosis rate of the selected A-series 2-indolinone derivative compounds is determined to be more than 20% compared to the control, which is determined as the success criterion for this work package. The determination of Annexin V/PI flow cytometry apoptosis values correlated with MTT results with an average of 40% and above proves that the tested compounds Y1, Y2, Y3, Y4, Y8 and Y14, whose general TKI activities are determined, show inhibitory effects by stimulating apoptosis.

Furthermore, tyrosine kinases are important regulatory molecules involved in cellular signal transduction that enables biological events such as cell proliferation, differentiation and migration. The compound of the present invention relates to a tyrosine kinase inhibitor and is provided for use in the treatment of the said cancers and the mechanism of action is achieved by inhibition of tyrosine kinases and induction of cell apoptosis.

In another preferred embodiments of the present invention, the binding energies to various RTKs (EGFR, HER2/neu VEGFR, PDGFR-β, IGFR, c-MET, AXL, CAK, FGFR4, HEK8, RET) are determined in molecular modeling studies performed with the compounds and it is determined that all of the compounds examined are effective at nanomolar level. The inventors have observed the highest binding to c-MET enzyme among the analyzed compounds. It is also revealed that the most effective derivative with a binding energy of less than −12 kcal/mol to the c-MET enzyme is the compound carrying chlorine at the 7-position of the indole ring.

In the light of these data, it can be seen that the compounds provided in the present invention for use in the treatment of human breast, lung, pancreas, colon and/or kidney cancer fulfill the need for new 2-indolinone derivatives in the relevant technical field. These compounds are particularly advantageous in that they are more effective, less toxic to healthy cells and have higher selectivity indices than sunitinib malate, which is one of the reference chemotherapy drugs currently used in the treatment.

The scope of protection of the invention is specified in the appended claims, and it is clear that a person skilled in the art can put forward similar embodiments in the light of what is explained above, without exceeding from the main theme of the invention.

1. A compound having the following formula for use in the treatment of human breast, lung, pancreas, colon and/or kidney cancer: 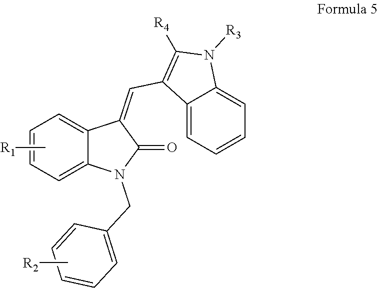
wherein;
R₁ is a hydrogen, alkyl, methoxy, trifluoromethoxy, halogen, nitro, cyano, carboxamide, sulfonic acid, sodium sulfonate or sulfonamide group;
R₂ is a hydrogen, alkyl, trifluoromethyl, methoxy, trifluoromethoxy, halogen, nitro, amine, dimethylamine, cyano, carboxylic acid, sulfonic acid or hydroxyl group; and
R₃ and R₄ are independently of each other a hydrogen or methyl group.

2. A compound for use according to claim 1, characterized in that the R₁, R₂, R₃ and R₄ groups are selected from the following groups:
R₁; H, 5-CH₃, 5-CH(CH₃)₂, 5-C(CH₃)₃, 5-OCH₃, 5-OCF₃, 5-F, 5-Cl, 5-Br, 5-I, 5-NH₂, 5-NO₂, 5-CN, 5-COOH, 5-CONH₂, 5-SO₃H, 5-SO₃Na, 5-SO₂NH₂, 7-CH₃, 7-CF₃, 7-OCH₃, 7-F, 7-Cl, 7-Br, 5,7-diCH₃, 5-F-7-CH₃, 5-Cl-7-CH₃, 7-F-6-Cl, 5,7-diCl, 5,7-diBr,
R₂; H, 2-CH₃, 2-C₂H₅, 2-CH(CH₃)₂, 2-C(CH₃)₃, 2-CF₃, 2-OCH₃, 2-SCH₃, 2-OCF₃, 2-F, 2-Cl, 2-Br, 2-I, 2-NO₂, 2-NH₂, 2-CN, 2-COOH, 2-OH, 3-CH₃, 3-C₂H₅, 3-CH(CH₃)₂, 3-C(CH₃)₃, 3-CF₃, 3-OCH₃, 3-SCH₃, 3-OCF₃, 3-SCF₃, 3-F, 3-Cl, 3-Br, 3-I, 3-NO₂, 3-NH₂, 3-CN, 3-COOH, 3-OH, 4-CH₃, 4-C₂H₅, 4-CH(CH₃)₃, 4-C(CH₃)₃, 4-CF₃, 4-OCH₃, 4-SCH₃, 4-OCF₃, 4-SCF₃, 4-F, 4-Cl, 4-Br, 4-I, 4-NO₂, 4-NH₂, 4-N(CH₃)₂, 4-CN, 4-COOH, 4-SO₃H, 4-OH, 2,3-diCH₃, 2,4-diCH₃, 2,5-diCH₃, 2,6-diCH₃, 3,4-diCH₃, 3,5-diCH₃, 3,5-diCF₃, 2,4-diOCH₃, 2,5-diOCH₃, 3,4-diOCH₃, 3,5-diOCH₃, 2-CH₃-3-Cl, 2-CH₃-5-Cl, 4-CH₃-3-CF₃, 2-OCH₃-5-CH₃, 2-F-5-CF₃, 4-F-3-CF₃, 2-Cl-5-CF₃, 4-Cl-3-CF₃, 5-Cl-2-OCH₃, 2,3-diF, 2,4-diF, 2,5-diF, 2,6-diF, 3,4-diF, 3,5-diF, 2,3-diCl, 2,4-diCl, 2,5-diCl, 2,6-diCl, 3,4-diCl, 3,5-diCl, 2-Cl-4F, 2-Cl-6E, 4-Br-2-F,
R₃; H or CH₃, and
R₄; H or CH₃.

3. The compound for use according to claim 1, characterized in that the compound is for use as a tyrosine kinase inhibitor and also for use as a compound for inducing cell apoptosis in cancer.
